(19) United States
(12) Patent Application Publication
(10) Pub. No.: US 2026/0146028 A1
(43) Pub. Date: May 28, 2026

(54) PROCESS FOR THE PREPARATION OF PYRAZOLE CARBOXYLIC ACID AMIDES

(71) Applicant: SYNGENTA CROP PROTECTION AG, Basel (CH)

(72) Inventors: Jihao SHAN, Nantong, Jiangsu (CN); Xin LIU, Nantong, Jiangsu (CN); Jianjian XUE, Nantong, Jiangsu (CN); Jinyang PAN, Nantong, Jiangsu (CN); Yaoping FENG, Nantong, Jiangsu (CN)

(73) Assignee: SYNGENTA CROP PROTECTION AG, Basel (CH)

(21) Appl. No.: 19/119,883

(22) PCT Filed: Oct. 11, 2022
(86) PCT No.: PCT/CN2022/124671

§ 371 Date: Apr. 10, 2025

(51) Int. Cl. C07D 231/14

(52) U.S. Cl. CPC C07D 231/14

(57) ABSTRACT

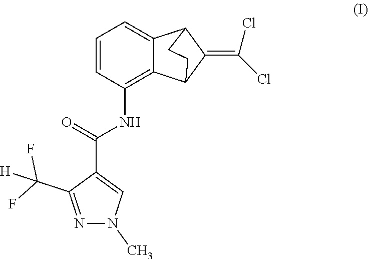 [SMILES: Cn1cc(C(=O)Nc2cccc3c2C2CCC3C2=C(Cl)Cl)c(C(F)F)n1]

The invention relates to a process for separating the solution mixture, characterized in that the solution mixture comprises a base, an organic solvent, water and the compound of formula (I) the process comprises the distillation of the solution mixture wherein there is a phase separator in the downstream of the distillation.

The present invention relates to a process for the preparation of pyrazole carboxylic acid amides, particularly 3-difluoromethyl-1-methyl-1H-pyrazole-4-carboxylic acid (9-dichloromethylene-1,2,3,4-tetrahydro-1,4-methano-naphthalen-5-yl)-amide, more particularly to a process for separating the solution mixture derived from its preparation.

The compound 3-difluoromethyl-1-methyl-1H-pyrazole-4-carboxylic acid (9-dichloromethylene-1,2,3,4-tetrahydro-1,4-methano-naphthalen-5-yl)-amide and its microbicidal properties is described for example in WO 2007/048556.

The preparation of 3-difluoromethyl-1-methyl-1H-pyrazole-4-carboxylic acid (9-dichloromethylene-1,2,3,4-tetrahydro-1,4-methano-naphthalen-5-yl)-amide is known from WO 2007/048556.

Said compound can be prepared according to schemes 1 and 4 by a) reacting the compound of formula A

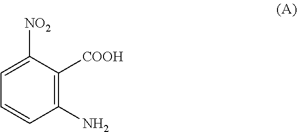 [SMILES: Nc1cccc([N+](=O)[O-])c1C(=O)O]

in the presence of an alkyl nitrite with a compound of formula B

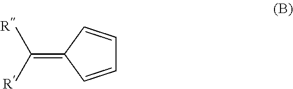

wherein R′ and R″ are e.g. C₁-C₄alkyl, to a compound of formula C

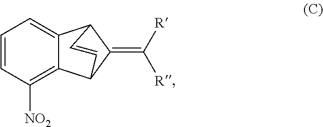

b) hydrogenating the compound of formula C in the presence of a suitable metal catalyst to a compound of formula D

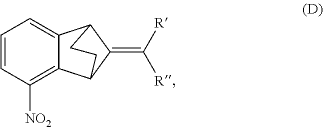

c) ozonising the compound of formula D and subsequent treatment with a reducing agent to a compound of formula E

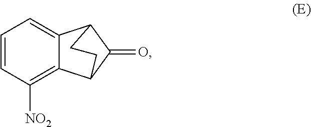 [SMILES: O=C1C2CCC1c1c2cccc1[N+](=O)[O-]]

d) reacting the compound of formula E in the presence of triphenylphosphane/carbon tetrachloride to 2, 9-dichloromethylidene-5-nitro-benzonorbornene of formula F

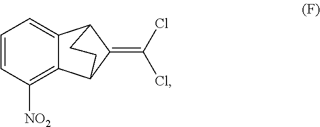 [SMILES: O=[N+]([O-])c1cccc2c1C1CCC2C1=C(Cl)Cl]

e) hydrogenating the compound of formula F in the presence of a metal catalyst to 2,9-dichloromethylidene-5-amino-benzonorbornene of formula G

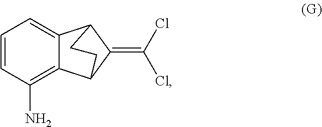 [SMILES: Nc1cccc2c1C1CCC2C1=C(Cl)Cl]

f) and reacting the compound of formula G with a compound of formula H

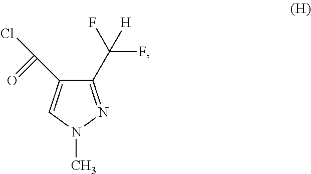

to 3-difluoromethyl-1-methyl-1H-pyrazole-4-carboxylic acid (9-dichloromethylene-1,2,3,4-tetrahydro-1,4-methano-naphthalen-5-yl)-amide.

More specifically, in the above-mentioned process for the preparation of 3-difluoromethyl-1-methyl-1H-pyrazole-4-carboxylic acid (9-dichloromethylene-1,2,3,4-tetrahydro-1,4-methano-naphthalen-5-yl)-amide (compound of formula (I)), xylene is used as a solvent for reaction, subsequent crystallization. In order to recycle the xylene, the solution must be purified by removing water and triethylamine (TEA). As a result, there is a part of xylene loss during the distillation.

Such loss should be reduced as low as possible for the cost and environmental consideration. Thus, the aim of the present application is to provide a novel process for increasing the recovery rate of such organic solvent.

Thus, according to the present invention, there is provided a process for separating the solution mixture, characterized in that the solution mixture comprises a base, an organic solvent, water and the compound of formula (I)

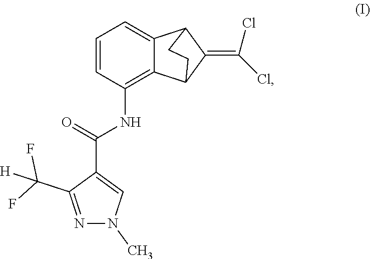 [SMILES: Cn1cc(C(=O)Nc2cccc3c2C2CCC3C2=C(Cl)Cl)c(C(F)F)n1]

the process comprising a distillation of the solution mixture wherein there is a phase separator in the downstream of the distillation.

According to a preferred embodiment of the present application, the base is triethylamine, pyridine, 2,6-lutidine, NaOH, KOH, sodium acetate, potassium carbonate, sodium carbonate, sodium bicarbonate, or potassium bicarbonate.

According to a preferred embodiment of the present application, the organic solvent is tetrahydrofuran, 1,4-dioxane, dimethoxyethane, diethyl ether, tert-butyl methyl ether, methyl-ethyl-ketone, ethyl acetate, methyl acetate, an aromatic hydrocarbon, or an aliphatic hydrocarbon.

According to a preferred embodiment of the present application, the organic solvent is an aromatic or aliphatic hydrocarbon.

According to a preferred embodiment of the present application, the organic solvent is toluene, xylene, benzene, hexane, pentane or a petroleum ether.

According to a preferred embodiment of the present application, there are at least one condenser, preferably two condensers in the downstream of the distillation.

According to a preferred embodiment of the present application, there are two condensers, in the downstream of the distillation.

According to a preferred embodiment of the present application, the phase separator is in the downstream of the condensers.

According to a preferred embodiment of the present application, the recovery rate of the organic solvent increases at least 15%, preferably at least 18%, more preferably at least 20% and most preferably at least 25%, in comparison with the condition without using the phase separator.

According to a preferred embodiment of the present application, the distillation is operated in batch mode.

According to a preferred embodiment of the present application, the content of the organic solvent in the solution mixture is from 65 wt % to 75 wt %, preferably from 67 wt % to 73 wt %, more preferably from 69 wt % to 71 wt %, over the total weight of the solution mixture.

According to the preferred embodiment of the present application, the content of the compound of formula (I) in the solution mixture is from 15 wt % to 25 wt %, preferably from 17 wt % to 23 wt %, more preferably from 19 wt % to 21 wt %, over the total weight of the solution mixture.

According to a preferred embodiment of the present application, the content of the base in the solution mixture is from 3 wt % to 7.5 wt %, preferably from 3.5 wt % to 7.3 wt %, more preferably from 3.9 wt % to 7.1 wt %, over the total weight of the solution mixture. According to a preferred embodiment of the present application, the content of water in the solution mixture is from 0.3 wt % to 4.5 wt %, preferably from 0.4 wt % to 4.3 wt %, more preferably from 0.5 wt % to 3.8 wt %, over the total weight of the solution mixture.

### DESCRIPTION OF DRAWINGS

FIG. 1 is the general flow chart for producing the compound of formula (I).

FIG. 2 is a prior art process for producing the compound of formula (I). without using phase separator.

FIG. 3 is the inventive process for producing the compound of formula (I), by using phase separator.

### PREFERRED EMBODIMENT

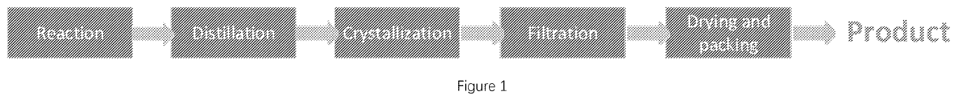

As indicated in FIG. 1, the desired compound is produced by a series reaction involving the use of the organic solvent, the base and water. After the reaction, there will be a solution mixture comprising the base, the organic solvent, water and the compound of formula (I) (identified as “Product” in FIG. 1). The volatile components, such as for example the base and/or the organic solvent, will be separated from the solution mixture through distillation. And the desired compound will be recovered by crystallization, filtration, drying and packing.

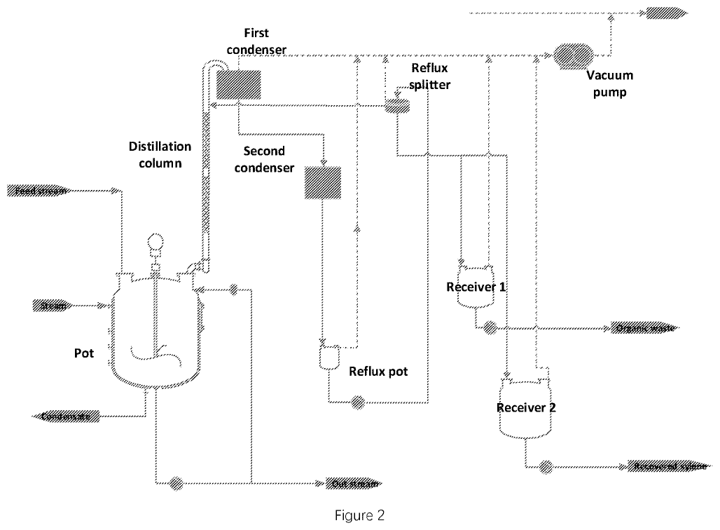

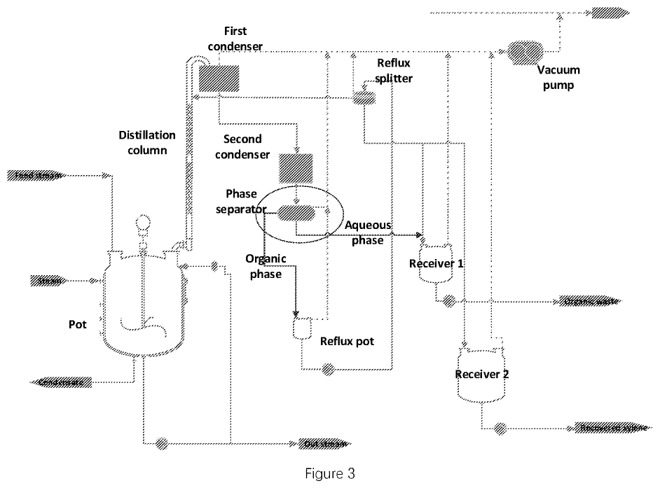

The distillation for separating such volatile components will be preferably operated in batch mode as shown in FIG. 2 and FIG. 3. The main difference between FIG. 2 and FIG. 3 is the use of the phase separator after distillation operation.

Such distillation can be performed in continuous, semi-continuous or batch, preferably batch mode. In the case of batch distillation, there are obtained two product streams, firstly the mixture of the organic solvent such as xylene and the base such as triethylamine having water content less than 0.05 wt %, and then the product stream mainly consisting of organic solvent such as xylene.

Generally, the operation condition of the distillation will be beneficial for the separation of the base, the organic solvent and water, particularly the distillation is operated under vacuum condition, such as 200 mbar to 400 mbar, preferably 250 mbar to 350 mbar (absolute pressure). The reflux ratio for the distillation will be from 1 to 5.

The function of the phase separator is mainly for the separation of water from the organic phase.

The resulting mixture recovered from the distillation can be recycled to the specific step for producing the compound of formula (I).

### PREPARATORY EXAMPLES

### Example 1 (Comparative)

In the distillation unit (FIG. 2), there are two fractions
1. 1000 kg head fraction in receiver 1 as organic waste which contain TEA (196 kg), xylene (706 kg) and 98 kg water.
2. Approx. 1255 kg high purity of xylene (TEA content <=0.3 wt. %, water content <=0.05 wt. %) as the second fraction in receiver 2 and be recovered back to process.

There is a lot of solvent loss in head fraction.

### Example 2 (Inventive)

After the improvement of distillation unit (FIG. 3), a new phase separator implemented in the system which could remove most water during distillation. As a result, more xylene and TEA recovered in second fraction with water content <=0.05 wt. %.

The new process of distillation has been implemented in the plant
1. Most water (78 kg) removed to receiver 1 (same vessel for head fraction) by phase separator.
2. 647 kg head fraction in receiver 1 as organic waste which contain TEA (176 kg), xylene (373 kg) and 20 kg water.
3. Approx. 1608 kg xylene and TEA mixture (water content <=0.05 wt. %) as the second fraction in receiver 2 and be recovered back to process.

The organic solvent recovery rate is increased by up to 28%.

1. A process for separating the solution mixture, characterized in that the solution mixture comprises a base, an organic solvent, water and the compound of formula (I) 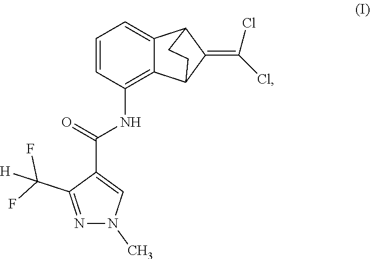 [SMILES: Cn1cc(C(=O)Nc2cccc3c2C2CCC3C2=C(Cl)Cl)c(C(F)F)n1]
the process comprising a distillation of the solution mixture wherein there is a phase separator in the downstream of the distillation.

2. The process according to the process of claim 1, characterized in that the base is triethylamine, pyridine, 2,6-lutidine, NaOH, KOH, sodium acetate, potassium carbonate, sodium carbonate, sodium bicarbonate, or potassium bicarbonate.

3. The process according to the process of claim 1, characterized in that the organic solvent is tetrahydrofuran, 1,4-dioxane, dimethoxyethane, diethyl ether, tert-butyl methyl ether, methyl-ethyl-ketone, ethyl acetate, methyl acetate, an aromatic hydrocarbon, or an aliphatic hydrocarbon.

4. The process according to the process of claim 1, characterized in that the organic solvent is an aromatic or aliphatic hydrocarbon.

5. The process according to the process of claim 1, characterized in that the organic solvent is toluene, xylene, benzene, hexane, pentane or a petroleum ether.

6. The process according to the process of claim 1, characterized in that there is at least one condenser, preferably two condensers, in the downstream of the distillation.

7. The process according to the process of claim 1, characterized in that there are two condensers in the downstream of the distillation.

8. The process according to the process of claim 6, characterized in that the phase separator is in the downstream of the condensers.

9. The process according to the process of claim 1, characterized in that the recovery rate of the organic solvent increases at least 15%, preferably at least 18%, more preferably at least 20% and most preferably at least 25%, in comparison with the condition without using the phase separator.

10. The process according to the process of claim 1, characterized in that the distillation is operated in batch mode.

11. The process according to the process of claim 1, characterized in that the content of the organic solvent in the solution mixture is from 65 wt % to 75 wt %, preferably from 67 wt % to 73 wt %, more preferably from 69 wt % to 71 wt %, over the total weight of the solution mixture.

12. The process according to the process of claim 1, characterized in that the content of the compound of formula (I) in the solution mixture is from 15 wt % to 25 wt %, preferably from 17 wt % to 23 wt %, more preferably from 19 wt % to 21 wt %, over the total weight of the solution mixture.

13. The process according to the process of claim 1, characterized in that the content of the base in the solution mixture is from 3 wt % to 7.5 wt %, preferably from 3.5 wt % to 7.3 wt %, more preferably from 3.9 wt % to 7.1 wt %, over the total weight of the solution mixture.

14. The process according to the process of claim 1, characterized in that the content of water in the solution mixture is from 0.3 wt % to 4.5 wt %, preferably from 0.4 wt % to 4.3 wt %, more preferably from 0.5 wt % to 3.8 wt %, over the total weight of the solution mixture.
